(19) United States
(12) Patent Application Publication
(10) Pub. No.: US 2026/0325250 A1
(43) Pub. Date: Oct. 22, 2026

(54) DEVICES FOR HOLDING A SURGICAL REMOTE

(71) Applicant: Alcon Inc., Fribourg (CH)

(72) Inventors: Brandon L. Spoelstra, Trabuco Canyon, CA (US); Daniel Durant, Rancho Santa Margarita, CA (US)

(21) Appl. No.: 19/468,084

(22) Filed: Feb. 3, 2026

Related U.S. Application Data
(60) Provisional application No. 63/763,393, filed on Feb. 26, 2025.

(51) Int. Cl. A61B 50/33

(52) U.S. Cl. CPC A61B 50/33

(57) ABSTRACT

Embodiments disclosed herein provide a cradle. The cradle includes a housing and one or more magnets disposed in the housing. The housing has an aperture therethrough for holding a remote that controls one or more aspects associated with operations of a surgical console. The one or more magnets are configured to magnetically interact with a magnetically attractive layer within a tray of the surgical console, and movably couple the housing to the tray. The housing further includes a first casing having a first surface configured to engage with the tray, and a second casing coupled to the first casing, the second casing having a second surface that faces an opposite direction of the first surface.

### INTRODUCTION

During various surgical procedures, a healthcare professional will use an array of instruments while operating on a patient. These instruments, together with certain supplies and consumables, are typically placed on a tray that is coupled to a surgical console. The tray is often positioned near the patient so that the items needed to complete the procedure are within reach of the healthcare professional. For example, a remote control device (also referred to as “remote”) for controlling one or more aspects of the surgical console may be placed on the tray so that the remote can easily be used by the healthcare professional.

Typically, during ophthalmic procedures, the tray and/or the remote may be covered with a surgical drape for sanitization purposes. However, in certain cases, the healthcare professional may accidentally touch or come into contact the remote during the procedure, which may cause the remote to transmit unwanted outputs, fall on its side, or even flip over, thereby making it more difficult to use. Further, when the surgical drape is removed and discarded after the procedure, the remote may fall off the tray and break or accidentally be discarded with the surgical drape.

### BRIEF SUMMARY

The present disclosure relates generally to devices for holding a remote during ophthalmic procedures.

In certain embodiments, a cradle is provided. The cradle includes a housing and one or more magnets disposed in the housing. The housing has an aperture therethrough for holding a remote that controls one or more aspects associated with operations of a surgical console. The one or more magnets are configured to magnetically interact with a magnetically attractive layer within a tray of the surgical console, and movably couple the housing to the tray.

In certain embodiments, a mount is provided. The mount includes a body and one or more attachment elements. The body has a cavity for holding a remote that controls one or more aspects associated with operations of a surgical console. The one or more attachment elements movably couple the body to a tray bail of the surgical console.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1A shows a top side isometric view of an example tray arm assembly with a cradle for holding a remote, according to certain embodiments.

FIG. 1B shows a top side isometric view of the tray arm assembly and the cradle without the remote of FIG. 1A, according to certain embodiments.

FIGS. 2A-2B show top and bottom side isometric views of the cradle of FIGS. 1A-1B, according to certain embodiments.

FIGS. 2C-2D show top and bottom side views of the cradle of FIGS. 2A-2B, according to certain embodiments.

FIGS. 2E-2F show exploded top and bottom side isometric views of the cradle of FIGS. 2A-2D, according to certain embodiments.

FIG. 3A shows another example cradle for coupling with a remote, according to certain embodiments.

FIG. 3B shows the cradle coupled with the remote of FIG. 3A, according to certain embodiments.

FIG. 4A shows a top side isometric view of the tray with the remote of FIG. 1A held by a mount, according to certain embodiments.

FIG. 4B shows a top side isometric view of the tray and the mount without the remote of FIG. 4A, according to certain embodiments.

FIG. 5A shows a top side isometric view of the mount of FIGS. 4A-4B, according to certain embodiments.

FIGS. 5B-5C show top and bottom side views of the mount of FIG. 5A, according to certain embodiments.

FIG. 5D shows an exploded top side isometric view of the mount of FIG. 5A, according to certain embodiments.

FIG. 6 shows a top side isometric view of the tray arm with the mount of FIGS. 4A-4B holding the remote in a different position, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

It will be readily understood that the components of the embodiments as generally described herein and illustrated in the appended Figures can be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in the Figures, the Figures are not necessarily drawn to scale unless specifically indicated.

Reference throughout this specification to the term “distal” refers to a system, device, component, end, portion, or segment that is disposed closer to a patient and/or further from a console during an ophthalmic procedure; and the term “proximal” refers to the system, device, component, end, portion, or segment that is disposed further from the patient and/or closer to the console during the ophthalmic procedure.

As discussed above, during surgical procedures, a tray is often used in operating rooms to hold various instruments, supplies, and/or consumables. For example, the tray may be used to support a remote control for controlling one or more aspects of a surgical console. The remote may be placed on the tray by a user (e.g., a surgeon or other healthcare professional) before the procedure and covered with a transparent or translucent surgical drape to maintain a sterile field and prevent contamination by the remote and/or the tray. The remote may then be manipulated by the user to control aspects of the surgical console during the procedure.

However, during such procedures, unwanted movement of the remote may occur when the remote is manipulated by the user. For example, the remote may slide, fall on its side, or flip over when the tray is moved or when the user makes accidental contact with the remote. Consequently, unwanted movement of the remote can have negative implications during surgical procedures. For example, such movement can make it difficult for a user to locate and/or manipulate one or more toggles or buttons on the remote, thereby impeding the performance of the surgical procedure. Further, such movement can cause toggles or buttons on the remote to be unintentionally manipulated, which can cause the surgical console to unintentionally perform certain functions.

Additionally, after surgery, when the surgical drape is removed and discarded during cleanup, the remote may be caught in, or picked up by, the surgical drape and accidentally thrown away with the surgical drape. This creates an unwanted expense because the remote may then need to be replaced.

The embodiments described herein provide remote holding devices that couple to a tray and hold the remote during ophthalmic procedures. Placing the remote in these holding devices allows for the remote to remain at a user-determined location and prevents the remote from accidentally sliding around or falling off the tray.

Accordingly, the remote holding devices described herein provide improved stability and flexibility during surgical procedures as compared to conventional systems. For example, the holding device may be placed at any user-determined location and orientation on the tray, and is not limited to placement at a predetermined and/or fixed location on the tray. Further, once placed in a location by the user, the holding device, which is coupled to the tray by one or more magnets or attachment elements, will remain in place until the user actively moves the holding device. In other words, the remote is less prone to unintentional and incidental movement because the holding device remains in place at the user-determined location. Thus, the user may be able to place and manipulate other instruments and/or devices on the tray, and carry out operations of the surgical procedure, without concern of accidentally moving the remote.

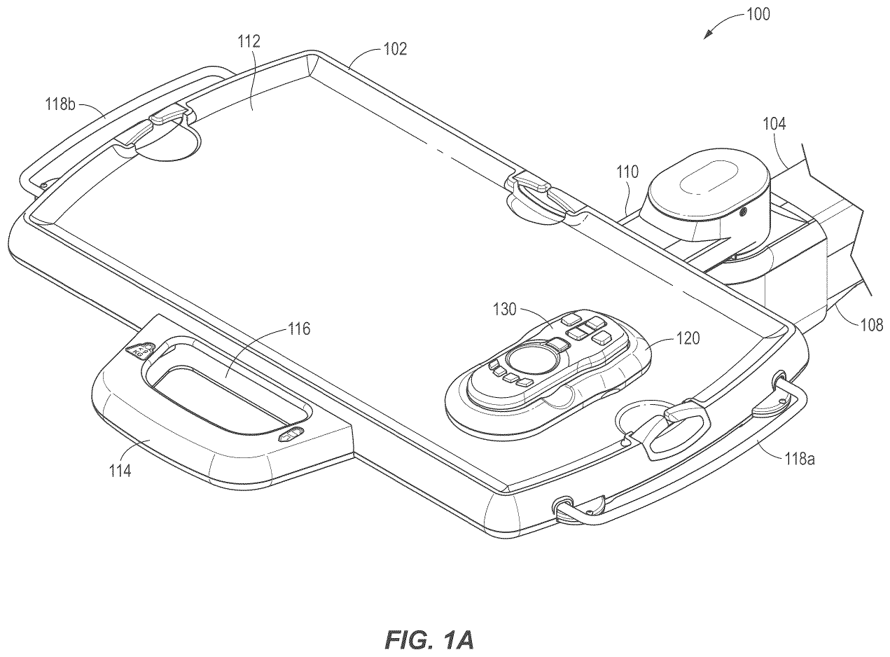

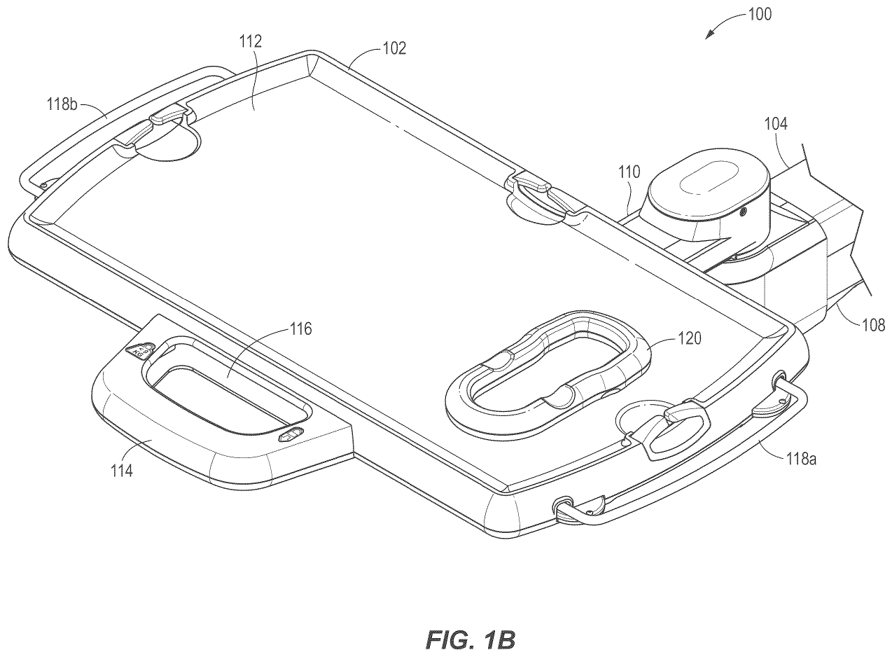

FIG. 1A shows a top side isometric view of an example tray arm assembly 100 with a cradle 120 for holding a remote 130, according to certain embodiments. FIG. 1B shows a top side isometric view of the tray arm assembly 100 and the cradle 120 without the remote 130 of FIG. 1A, according to certain embodiments. Accordingly, FIGS. 1A-1B are described together herein for clarity purposes.

The tray arm assembly 100 is constructed to be an integral part of a surgical console. As shown, the tray arm assembly 100 comprises a tray 102, an upper arm assembly 104, an elbow joint 108, a forearm assembly 110, a tray positioning handle 114, and a release handle 116. The tray 102 includes an instrument surface 112 with the capacity to hold instruments, equipment, supplies, and/or any consumables required for a surgical procedure, in addition to any associated cables and tubes. The tray 102 further includes two tray bails 118a, 118b (118a-b) which may extend or slide outwards from the tray 102. In certain embodiments, the tray arm assembly 100 may also include a shoulder joint, a tray joint, and a mount for connecting the tray arm assembly 100 to the surgical console.

The elbow joint 108, the shoulder joint, and the tray joint are configured to allow the tray 102 to be positioned (e.g., moved or rotated) with respect to the surgical console. Also, one or more of the elbow joint 108, the shoulder joint, and the tray joint comprise a two-tier friction mechanism. The two-tier friction mechanism comprises a first friction mechanism and a second friction mechanism. If activated, the first friction mechanism prevents any movement in the corresponding joint unless enough torque is applied to the joint to overcome the friction associated with the first friction mechanism. In other words, when the first friction mechanism is activated in all the joints, a user is not able to move or rotate the tray arm assembly 100 relative to the surgical console unless the user applies enough torque to overcome the first friction mechanism in one or more of the joints. Once enough torque is applied to a joint and the first friction mechanism is overcome, the corresponding components that are coupled to the joint can be moved or rotated relative to the surgical console.

By grabbing and pulling the release handle 116, a user is able to deactivate the first friction mechanism in every joint with a first friction mechanism in order to position the tray 102 with respect to the surgical console. However, even with the first friction mechanism de-activated, each joint is still subject to a second friction mechanism, which is always active in the joint. The second friction mechanism subjects each joint to a drag torque, which prevents the whipping of the tray arm assembly 100 relative to the surgical console. Drag torque is exerted in each joint by the second friction mechanism in the opposite direction of torque applied to the joint. Note that applying torque to a joint herein may include applying torque to one or more of the components that are coupled to the joint. For example, applying torque to the elbow joint 108 includes or can be caused by applying torque to the forearm assembly 110 and the upper arm assembly 104. In another example, applying torque to the shoulder joint includes applying torque to the forearm assembly 110 and the surgical console.

By releasing the release handle 116, a user is able to re-activate the first friction mechanism in the joints. When the first friction mechanism is activated, an application of force to one or more components of the tray arm assembly 100 does not cause any movement or rotation in the tray arm assembly 100 relative to the surgical console, unless such a force results in torque exceeding a threshold amount of torque required to overcome the first friction mechanism in one or more of the joints. As such, when the first friction mechanism is activated in the joints, a user is able to grab the handle 114 and move the surgical console by pushing and pulling the handle 114 or any other part of the tray 102, the upper arm assembly 104, the forearm assembly 110, etc.

Note that the tray arm assembly 100 described herein is entirely mechanical, however, those of ordinary skill in the art will understand that in an alternate embodiment, one or more powered actuators may be used to move the components, including the arm assemblies and joints of tray arm assembly 100.

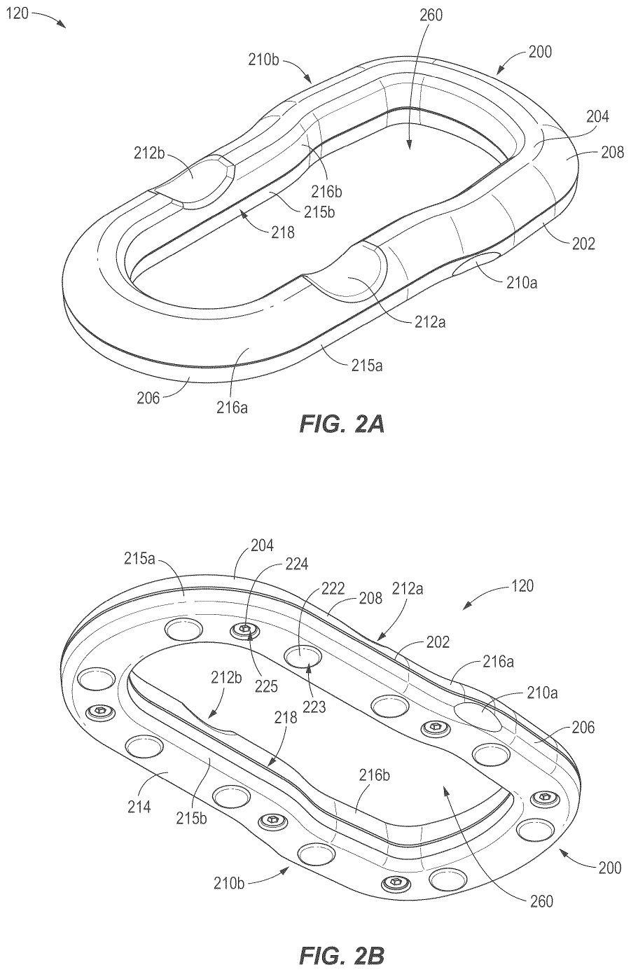

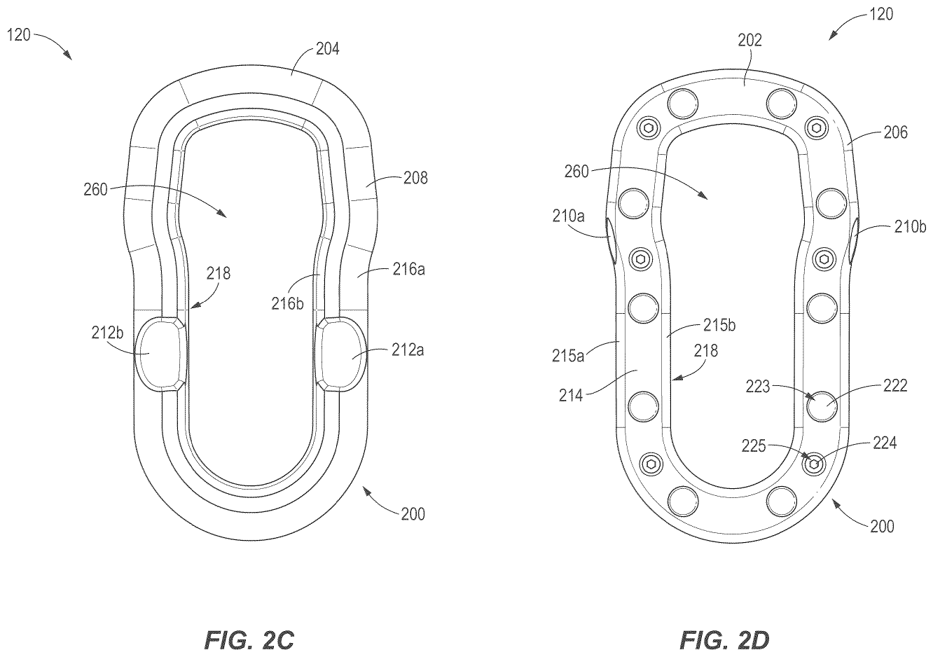

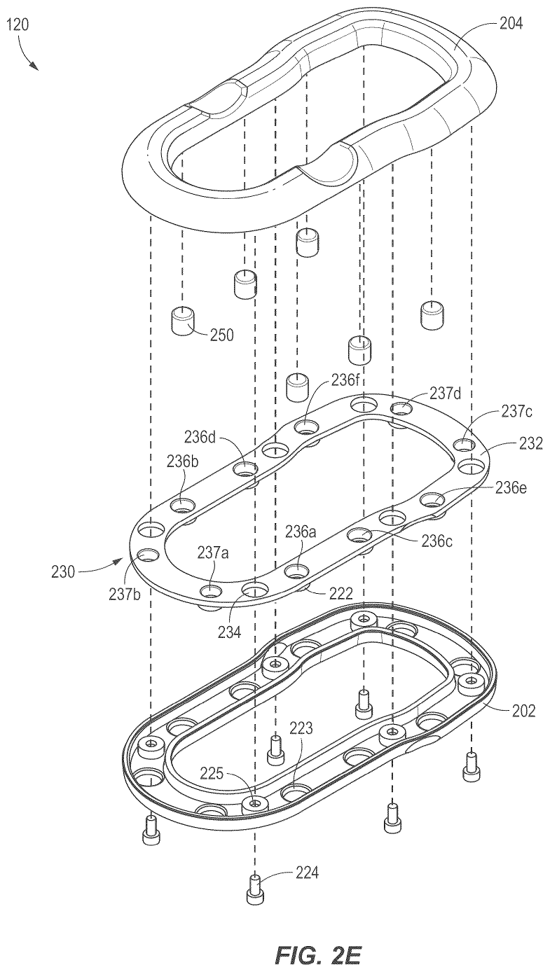

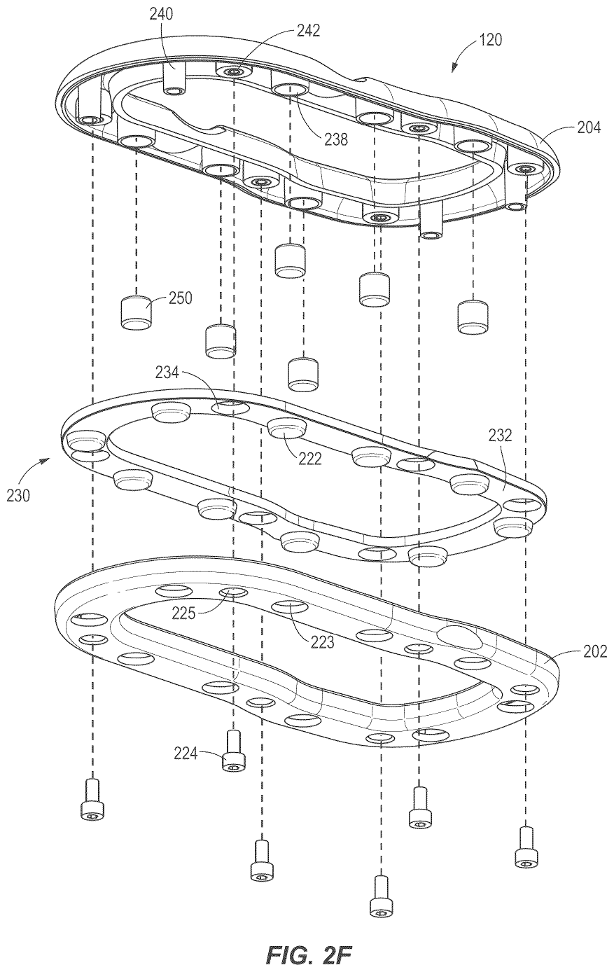

The tray arm assembly 100 is configured to support, in addition to the other equipment, a cradle 120 and a remote 130 that controls one or more operations of the surgical console. The cradle 120 magnetically couples to the tray 102 at the user-determined location as described in further detail with respect to FIGS. 2E-2F. Once the cradle 120 is positioned at a user-determined location, the remote 130 is placed in the cradle 120. The remote 130 may also magnetically couple to the tray 102. The tray 102, the cradle 120, and the remote 130 may then be covered with a transparent or translucent surgical drape to maintain a sterile field and prevent contamination by the tray 102, the cradle 120, and the remote 130. In some embodiments, the cradle 120 and remote 130 may be a single/unitary piece (the cradle may be incorporated into the perimeter case of the remote 130).

As described in further detail with respect to FIGS. 2A-2D, the cradle 120 maintains positioning of the remote 130 on the tray 102 and prevents the remote 130 from accidentally sliding, falling on its side, or flipping over on the tray 102. In other words, the cradle 120 prevents unwanted movement of the remote 130 during surgery. Further, the cradle 120 helps keep the remote 130 on the tray 102 when the surgical drape is removed after surgery.

FIGS. 2A-2B show top and bottom side isometric views of the cradle 120 of FIGS. 1A-1B, according to certain embodiments. FIGS. 2C-2D show top and bottom side views of the cradle 120 of FIGS. 2A-2B, according to certain embodiments. Accordingly, FIGS. 2A-2D are described together herein for clarity purposes.

The cradle 120 includes a housing 200 that defines an aperture 260 for holding a remote (e.g., remote 130). The housing 200 of the cradle 120 includes a first casing 202 and a second casing 204. The first casing 202 is coupled to the second casing 204 by six screws 224 (one labeled for brevity). The first casing 202 and the second casing 204 define an inner perimeter 218 of the housing 200 that engages with one or more surfaces of the remote 130.

The first casing 202 has a first surface 206 configured to engage with a tray (e.g., tray 102). That is, the first surface 206 faces the instrument surface 112 of the tray 102 when the cradle 120 is disposed on the instrument surface 112. The first surface 206 includes a planar portion 214, a first convex portion 215a, and a second convex portion 215b. The planar portion 214 is between the first convex portion 215a and the second convex portion 215b. The curvature or roundness of the first convex portion 215a allows the user to grip the underside of the cradle 120 when lifting or moving the cradle 120 relative to a tray (e.g., tray 102).

The first casing 202 further includes a first set of two depressions 210a, 210b (210a-b) formed in the first convex portion 215a of the first surface 206. The first set of two depressions 210a-b are horizontally aligned with each other on opposite sides of the first surface 206 and extend at least partially inwards towards the aperture 260. The first set of two depressions 210a-b of the first casing 202 help further facilitate easy removal of the cradle 120 from the tray 102 when the cradle 120 is coupled to the tray 102 as shown in FIG. 1B. For example, the user can grip the cradle 120 at the first set of two depressions 210a-b to lift the cradle 120 off the instrument surface 112 of the tray 102. The first set of two depressions 210a-b of the first casing 202 may also be used to reposition the cradle 120 on the tray 102. For example, the user can hold the cradle 120 at the first set of two depressions 210a-b and slide the cradle 120 on the instrument surface 112 of the tray 102 to a different position.

The first casing 202 also defines ten elastomeric pad openings 223 (one labeled for brevity) and six screw openings 225 (one labeled for brevity). The elastomeric pad openings 223 are disposed equidistantly from each other around the first casing 202. Ten elastomeric pads 222 (one labeled for brevity) protrude through the elastomeric pad openings 223 from within the housing 200 and help maintain positioning of the housing 200 on the tray 102 at a user-determined location. The screws 224 extend through the screw openings 225 to couple the first casing 202 to the second casing 204. The elastomeric pad openings 223, screw openings 225, elastomeric pads 222, and screws 224 are best seen in FIGS. 2E-2F.

The second casing 204 has a second surface 208 that faces an opposite direction of the first surface 206. That is, the second surface 208 faces away from the instrument surface 112 of the tray 102 when the cradle 120 is disposed on the instrument surface 112. The second surface 208 has a convex shape formed by a first convex portion 216a and a second convex portion 216b. The curvature or roundness of the first convex portion 216a prevents a surgical drape from catching or snagging edges of the remote 130 held by the cradle 120 (e.g., as shown in FIG. 1A) when the surgical drape is pulled over the remote 130 and the tray 102 before surgery, or discarded after surgery. In other words, the first convex portion 216a reduces unwanted movement of the remote 130 as caused by unintentional pulling on the remote 130 by the surgical drape, and helps stop the remote 130 from accidentally being thrown away during cleanup.

The second casing 204 further includes a second set of two depressions 212a, 212b (212a-b) formed in the second surface 208 between the first convex portion 216a and the second convex portion 216b. The second set of two depressions 212a-b are horizontally aligned with each other on opposite sides of the second surface 208 and extend at least partially towards the first casing 202. The second set of two depressions 212a-b of the second casing 204 allow for easy insertion and removal of the remote 130 into and from the cradle 120. For example, the second set of two depressions 212a-b of the second casing 204 allow the user to hold the remote 130 until the remote 130 is positioned in the cradle 120 and in contact with the instrument surface 112 (i.e., coupled with the tray 102) as shown in FIG. 1A. When the user wants to remove the remote 130 from the cradle 120 after the remote 130 is placed in the cradle 120, the user can grip outer portions of the remote 130 that are exposed by the second set of two depressions 212a-b and lift the remote 130 off the instrument surface 112 of the tray 102.

FIGS. 2E-2F show exploded top and bottom side isometric views of the cradle 120 of FIGS. 2A-2D, according to certain embodiments.

As shown in FIG. 2E, the cradle 120 further comprises an elastomeric element 230 and six magnets 250 (one labeled for brevity). The elastomeric element 230 is disposed between the first casing 202 and the second casing 204. The elastomeric element 230 comprises an elastomeric ring 232 and the elastomeric pads 222 that protrude outwards from the elastomeric ring 232. The elastomeric ring 232 defines six screw openings 234 (one labeled for brevity). The elastomeric pads 222 define six magnet receptacles 236a, 236b, 236c, 236d, 236e, 236f (236a-f) and four post receptacles 237a, 237b, 237c, 237d (237a-d).

As shown in FIG. 2F, the second casing 204 further includes six receptacles 238 (one labeled for brevity), four posts 240 (one labeled for brevity), and six receivers 242 (one labeled for brevity).

In view of FIGS. 2E-2F, when the first casing 202 is coupled to the second casing 204, the magnets 250 are disposed in the magnet receptacles 236a-f of the elastomeric element 230 and the receptacles 238 of the second casing 204. In certain embodiments, the magnets 250 may be press fit into the receptacles 238 or held in the receptacles 238 by glue, a screw, or other suitable coupling mechanism. As an example, the magnets 250 may be neodymium magnets. The magnets 250 are configured to magnetically interact with a magnetically attractive layer disposed within a tray (e.g., tray 102) of a surgical console, and movably couple the cradle 120 to the tray 102.

Additionally, when the first casing 202 is coupled to the second casing 204, the posts 240 are disposed in the post receptacles 237a-d, and the screws 224 extend through the screw openings 234 to couple with the receivers 242. The elastomeric pads 222 of the elastomeric element 230 also protrude through the elastomeric pad openings 223 of the first casing 202. The elastomeric pads 222 have a thickness that permits magnetic coupling of the cradle 120 to a tray (e.g., tray 102), while still preventing the cradle 120 from sliding around when disposed on the tray 102.

Although the cradle 120 is shown in FIGS. 2A-2F as having a certain shape, the cradle 120 may also have a different shape based on differently shaped surgical remotes. The cradle 120 is also not limited to the number and/or arrangement of each component shown in FIGS. 2A-2F. For example, the cradle 120 may include a different number and/or arrangement of elastomeric pads 222, elastomeric pad openings 223, screws 224, screw openings 234, receptacles 238, posts 240, receivers 242, and/or magnets 250.

Similarly, the first casing 202 and/or the second casing 204 may have zero depressions, one depression, or more than two depressions. The depressions on the first casing 202 and/or the second casing 204 may also have a different shape and/or be positioned at different locations on the first casing 202 and/or the second casing 204. In some embodiments, one or more depressions may be shared by both the first casing 202 and the second casing 204.

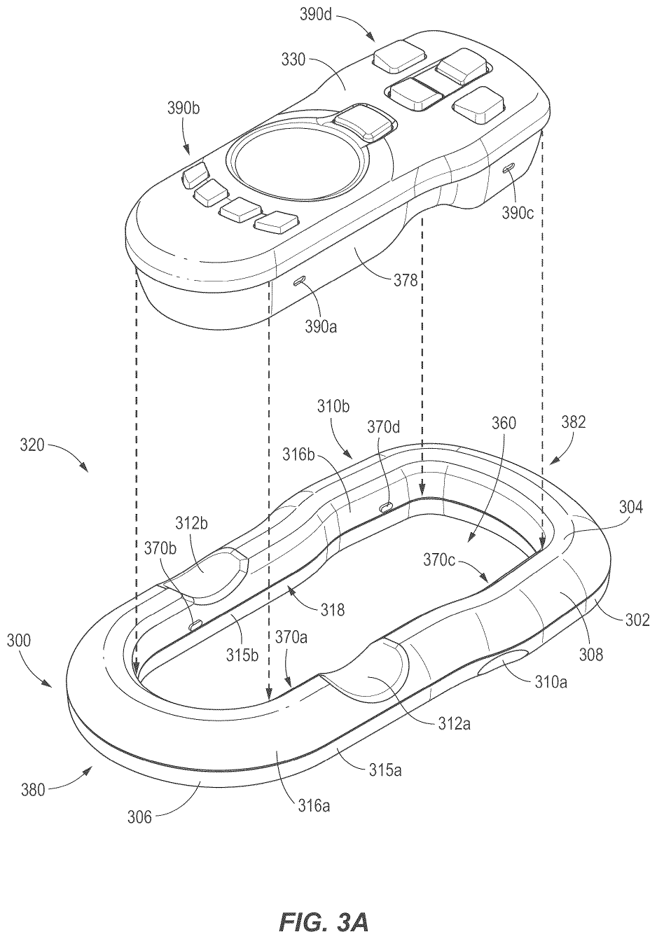

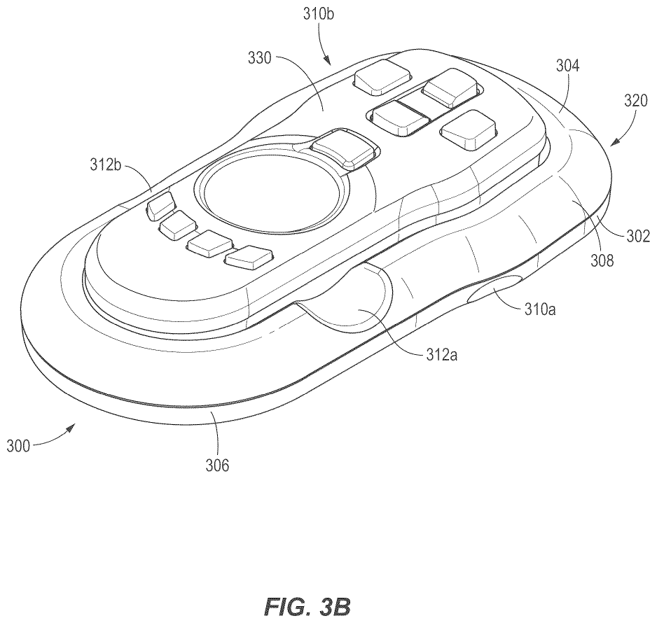

FIG. 3A shows another example cradle 320 for coupling with a remote 330, according to certain embodiments. Similar to the cradle 120 described with reference to FIGS. 2A-2F, the cradle 320 shown in FIG. 3A also includes a housing 300 defining an inner perimeter 318 and an aperture 360. The housing 300 includes a first casing 302 having a first surface 306 and a second casing 304 having a second surface 308. The first surface 306 includes a first convex portion 315a, a second convex portion 315b, and a first set of two depressions 310a, 310b (310a-b). The second surface 308 includes a first convex portion 316a, a second convex portion 316b, and a second set of two depressions 312a, 312b (312a-b).

As shown in FIG. 3A, the cradle 320 further includes four coupling elements 370a, 370b, 370c, 370d (370a-d) for coupling the cradle 320 with the remote 330. The coupling elements 370a-d are disposed on the second convex portion 316b of the second casing 304. Two of the coupling elements 370a, 370b are horizontally aligned with each other towards a first end 380, while the other two coupling elements 370c, 370d are horizontally aligned with each other towards a second end 382. As an example, the coupling elements 370a-d protrude laterally from the second casing 304. In other words, the coupling elements 370a-d extend inwards toward the aperture 360.

The remote 330 is similar to the remote 130 shown in FIG. 1A, but further includes an outer surface 378 with four receiving elements 390a, 390b, 390c, 390d (390a-d). The receiving elements 390a-d are configured to receive the coupling elements 370a-d of the cradle 320. In certain embodiments, the receiving elements 390a-d may be indentations or apertures formed in the outer surface 378 of the remote 330. When coupling the remote 330 with the cradle 320, the coupling elements 370a-d of the cradle 320 click or snap into the receiving elements 390a-d of the remote 330.

FIG. 3B shows the cradle 320 coupled with the remote 330 of FIG. 3A, according to certain embodiments. Once the cradle 320 is coupled with the remote 330, the cradle 320 and the remote 330 can be positioned on a tray (e.g., tray 102) together. Similarly, the cradle 320 and the remote 330 can be collectively repositioned on the tray 102 and/or removed from the tray 102. Because the cradle 320 is coupled with the remote 330, the user may move the remote 330 by moving the cradle 320. Further, the cradle 320 can be moved without the remote 330 accidentally falling out of the cradle 320.

The remote 330 may be removed from the cradle 320 if the user lifts the remote 330 up while holding the cradle 320. For example, when the user pulls the remote 330 out of the cradle 320, the coupling elements 370a-d uncouple from the receiving elements 390a-d, allowing the remote 330 to be separated from cradle 320. In other words, the coupling elements 370a-d of the cradle 320 click or snap out of the receiving elements 390a-d of the remote 330.

Although the cradle 320 is shown in FIG. 3A as including four coupling elements 370a-d, the cradle 320 may also have less than or more than four coupling elements. The coupling elements 370a-d may also have a different shape and/or be positioned at different locations on the housing 300 so long as the coupling elements 370a-d correspond with a shape and positioning of the receiving elements 390a-d of the remote 330. In some embodiments, one or more coupling elements may be shared by both the first casing 302 and the second casing 304.

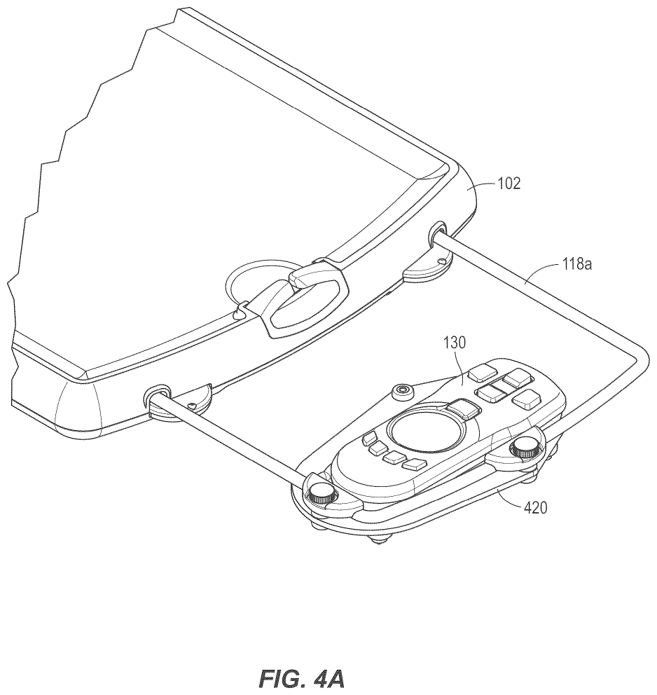

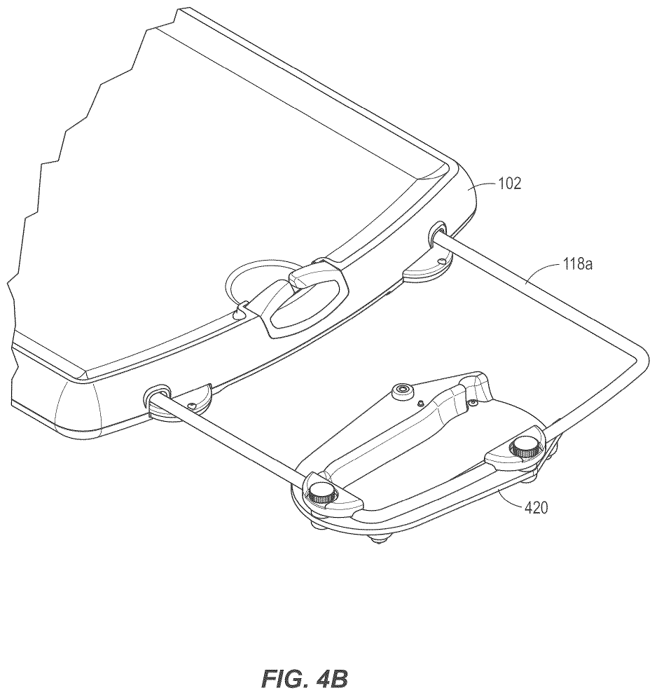

FIG. 4A shows a top side isometric view of the tray 102 with the remote 130 of FIG. 1A held by a mount 420, according to certain embodiments. FIG. 4B shows a top side isometric view of the tray 102 and the mount 420 without the remote 130 of FIG. 4A, according to certain embodiments. Accordingly, FIGS. 4A-4B are described together herein for clarity purposes.

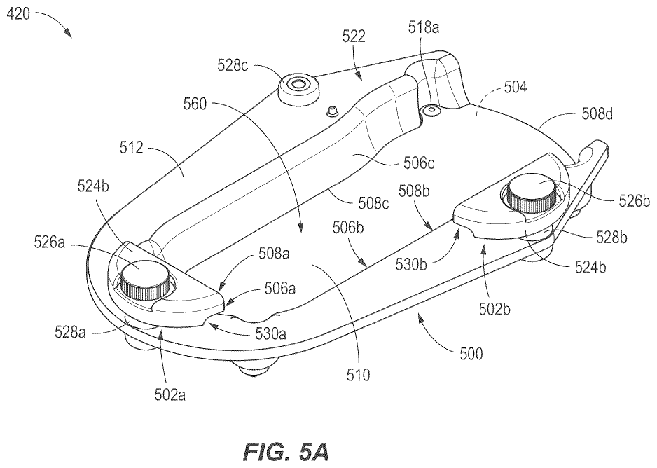

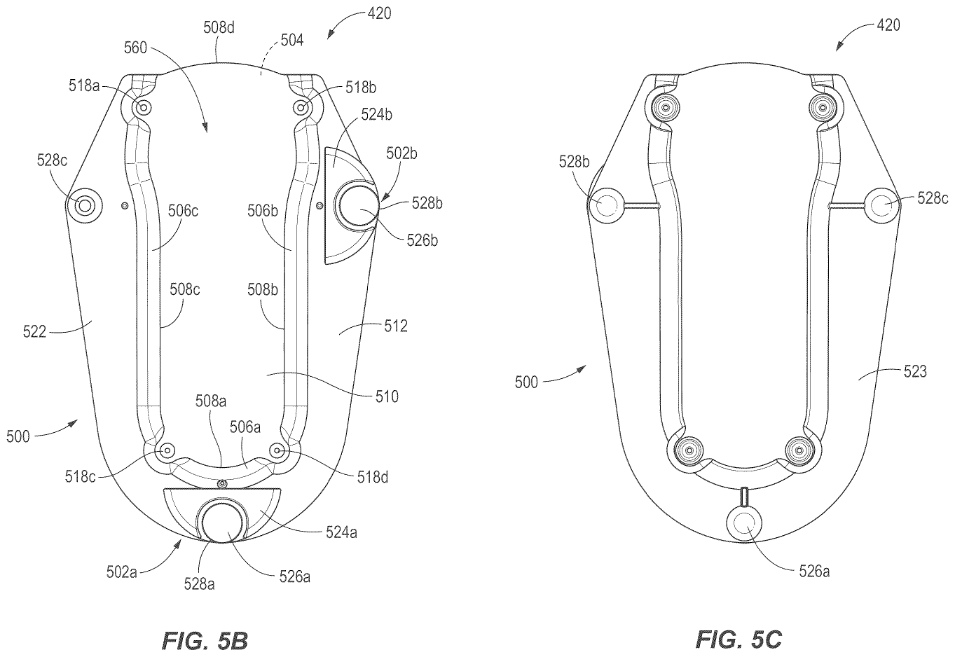

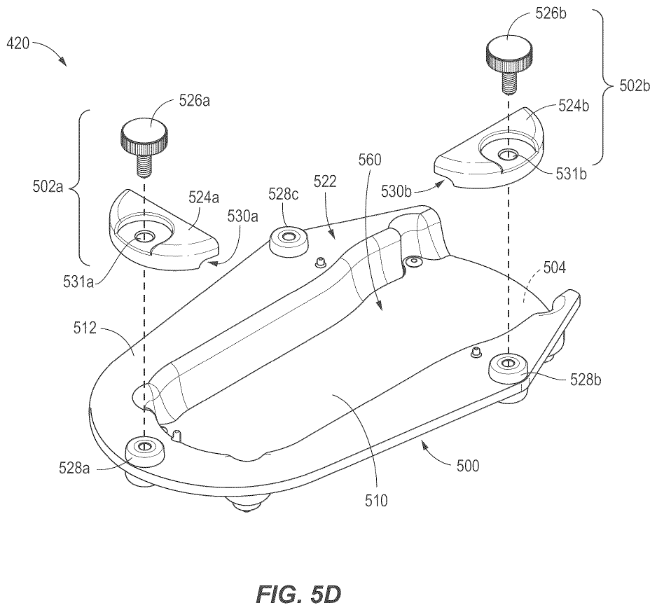

As shown in FIGS. 4A-4B, the tray bail 118a is an extended position, such that the tray bail 118a has been pulled out of the tray 102. The tray bail 118a is configured to support, in addition to the other equipment, the mount 420 and the remote 130 that controls one or more aspects of a surgical console. The mount 420 movably couples to the tray bail 118a at a user-determined location as described in further detail with respect to FIGS. 5A-5D. Once the mount 420 is positioned at the user-determined location, the remote 130 is placed in the mount 420. The tray 102, the mount 420, and the remote 130 may then be covered with a transparent or translucent surgical drape to maintain a sterile field and prevent contamination by the tray 102, mount 420, and the remote 130. In some embodiments, the mount 420 and remote 130 may be a single/unitary piece (the mount may be incorporated into a perimeter case of the remote 130).

By coupling the mount 420 to the tray bail 118a, the user may have more space on the tray 102 because the remote 130 can be held next to the tray 102 by the mount 420. Thus, the user may be able to place other instruments and/or devices on the tray, and carry out operations of the surgical procedure, without concern of accidentally moving the remote. As described in further detail with respect to FIGS. 5A-5D, the mount 420 helps maintain positioning of the remote 130 relative to the tray 102 and prevents unwanted movement of the remote 130 during surgery. Further, the mount 420 helps hold the remote 130 in place when the surgical drape is removed after surgery.

FIG. 5A shows a top side isometric view of the mount 420 of FIGS. 4A-4B, according to certain embodiments. FIGS. 5B-5C show top and bottom side views of the mount of FIG. 5A, according to certain embodiments. FIG. 5D shows an exploded top side isometric view of the mount 420 of FIG. 5A, according to certain embodiments. Accordingly, FIGS. 5A-5D are described together herein for clarity purposes.

The mount 420 includes a body 500 that defines a cavity 560 for holding a remote (e.g., remote 130). The body 500 of the mount 420 includes a base surface 504 and three walls 506a, 506b, 506c (506a-c) that define the cavity 560 and engage with one or more surfaces of a remote (e.g., remote 130). The base surface 504 includes four edges 508a, 508b, 508c, 508d (508a-d). The walls 506a-c are disposed around a portion of the base surface 504, such that walls 506a-c are disposed along three of the four edges 508a-d of the base surface 504. Particularly, the walls 506a, 506b, 506c, are disposed along the edges 508a, 508b, 508c, respectively. The edge 508d is exposed, or does not have a wall formed therealong, so that the user can easily pull the remote 130 out of the mount 420.

The mount 420 further includes a magnetically attractive layer 510 disposed in the cavity 560 and configured to magnetically interact with one or more magnets within a remote (e.g., remote 130) to couple the remote 130 to the body 500. The magnetically attractive layer 510 is coupled to the base surface 504 by four rivets 518a, 518b, 518c, 518d (518a-d). The magnetically attractive layer 510 matches a shape of the base surface 504. Examples of the magnetically attractive layer 510 include a ferrous sheet, steel sheet, or other similar magnetic sheet.

When the remote 130 is placed in the mount 420 (seen in FIG. 4A), the magnet(s) within the remote 130 magnetically couple to the magnetically attractive layer 510. The magnetic coupling between the remote 130 and the mount 420, in combination with the walls 506a-c, prevent the remote 130 from falling out of the mount 420. For example, the walls 506a-c and the magnetically attractive layer 510 prevent unwanted movement of the remote 130 when a surgical drape is pulled over the remote 130 and the tray 102 before surgery, or discarded after surgery. In other words, the magnetically attractive layer 510 reduces unwanted movement of the remote 130 as caused by unintentional pulling on the remote 130 by the surgical drape, and helps stop the remote 130 from accidentally being thrown away during cleanup. The walls 506a-c and the magnetically attractive layer 510 also prevent unwanted movement of the remote 130 when the user makes accidental contact with the remote 130.

Turning back to FIGS. 5A-5C, the body 500 further includes a mounting portion 512 protruding outwards from the walls 506a-c. The mounting portion 512 has a first surface 522 that faces the tray bail 118a, and a second surface 523 that is opposite the first surface 522. Three receiving elements 528a, 528b, 528c (528a-c) are formed on the first surface 522 of the mounting portion 512. The receiving elements 528a-c are arranged in a triangular shape around the body 500, such that one receiving element 528a is disposed between two receiving elements 528b, 528c that are horizontally aligned with each other. The receiving elements 528a-c are configured to receive attachment elements, such as attachment elements 502a, 502b (502a-b) best seen in FIG. 5D.

Turning to FIG. 5D, the attachment elements 502a-b movably couple the body 500 to a tray bail (e.g., tray bail 118a) of a surgical console. The attachment elements 502a, 502b are comprised of clips 524a, 524b (clips 524a-b) and screws 526a, 526b (526a-b). The clips 524a, 524b each include a curved surface 530a, 530b (530a-b), respectively, configured to engage with the tray bail 118a. Additionally, the clips 524a, 524b each include an aperture 531a, 531b (531a-b), respectively, through which the screws 526a-b extend to couple the clips 524a-b to the body 500.

In view of FIGS. 4A-4B and FIGS. 5A-5D, to couple the mount 420 to the tray bail 118a, the screws 526a-b are first loosened so that the clips 524a-b can slide over the tray bail 118a at a user-determined position. Once the mount 420 is at the user-determined position, the screws 526a-b are tightened to securely clamp the mounting portion 512 of the body 500 and the clips 524a-b of the attachment elements 502a-b against the tray bail 118a. For example, the first surface 522 of the mounting portion 512 engages with a first side of the tray bail 118a, and the curved surfaces 530a-b of the clips 524a-b engage with a second side of the tray bail 118a. When the mount 420 is coupled to the tray bail 118a, the base surface 504 and the first surface 522 are parallel to the tray bail 118a.

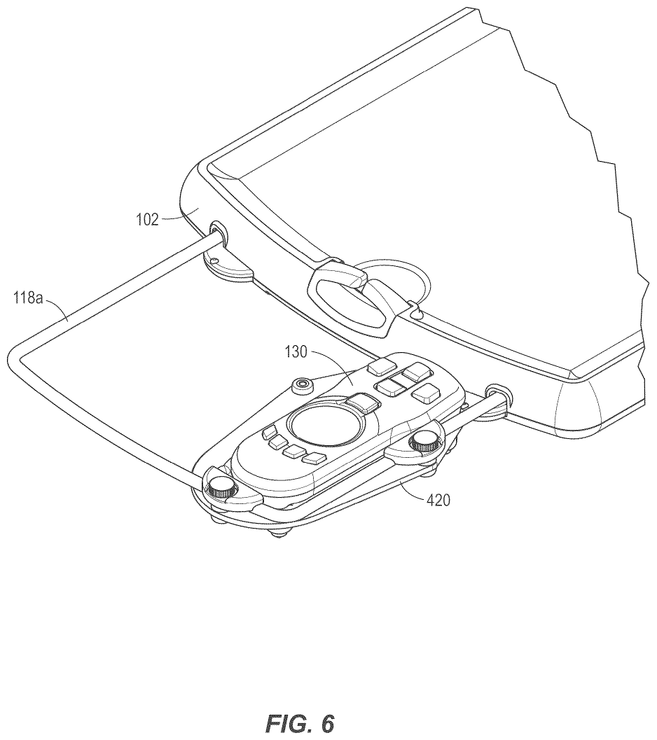

By coupling the mount 420 to the tray bail 118a via the attachment elements 502a-b, the mount 420 can be movably coupled to the tray bail 118a at various user-determined positions. As such, the user can couple the mount 420 to the tray bail 118a based on their preferences or the procedure being performed. As an example, if the user prefers to couple the mount 420 on the other side of the tray 102 (e.g., to tray bail 118b shown in FIGS. 1A-1B), then the user can move the attachment element 502b to couple with the receiving element 528c. As another example, if the user prefers to couple the mount 420 to a different location on the tray bail 118a, the user can loosen the screws 526a-b, move the mount 420, and retighten the screws 526a-b at the different location. Such an example is described in further detail with respect to FIG. 6.

Although the mount 420 is described as including the magnetically attractive layer 510 to couple the remote 130 to the body 500, in certain embodiments, the mount 420 may not include the magnetically attractive layer 510. In such embodiments, the walls 506a-c of the mount 420 hold the remote in place. In certain other embodiments, instead of the magnetically attractive layer 510, the mount 420 may include coupling elements similar to the coupling elements 370a-d shown in FIG. 3A. For example, the mount 420 may include such coupling elements on one or more of the walls 506a-c, so long as a remote has receiving elements similar to the receiving elements 390a-d of the remote 330.

Further, although the mount 420 is shown in FIGS. 5A-5D as having a certain shape, the mount 420 may also have a different shape based on differently shaped surgical remotes. The mount 420 is also not limited to the number and/or arrangement of each component shown in FIGS. 5A-5D. For example, the mount 420 may include a different number and/or arrangement of attachment elements, receiving elements, and/or walls.

FIG. 6 shows a top side isometric view of the tray 102 with the mount 420 of FIGS. 4A-4B holding the remote 130 in a different position, according to certain embodiments. As shown in FIG. 6, the mount 420 is positioned in a different corner of the tray bail 118a, for example, relative to the position of the mount 420 seen in FIGS. 4A-4B. The mount 420 may also be positioned in various other user-determined locations as noted above.

The present disclosure may be embodied in other specific forms without departing from its spirit or essential characteristics. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to “one embodiment”, “an embodiment”, or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

### Example Embodiments

Embodiment 1: A mount, the mount comprising: a body having a cavity for holding a remote that controls one or more aspects associated with operations of a surgical console; and one or more attachment elements for movably coupling the body to a tray bail of the surgical console.

Embodiment 2: The mount of Embodiment 1, wherein the body comprises: a base surface; and one or more walls disposed around a portion of the base surface, wherein the one or more walls and the base surface define the cavity.

Embodiment 3: The mount of Embodiment 2, wherein the base surface comprises four edges and the one or more walls are disposed along three of the four edges.

Embodiment 4: The mount of Embodiment 2, wherein the one or more walls and the base surface engage with one or more surfaces of the remote.

Embodiment 5: The mount of Embodiment 2, wherein the base surface is parallel to the tray bail.

Embodiment 6: The mount of Embodiment 2, wherein the body further comprises: a mounting portion protruding outwards from the one or more walls.

Embodiment 7: The mount of Embodiment 6, wherein the mounting surface is parallel to the tray bail.

Embodiment 8: The mount of Embodiment 1, further comprising: a magnetically attractive layer disposed in the cavity and configured to magnetically interact with one or more magnets within the remote to couple the remote to the body.

Embodiment 9: The mount of Embodiment 8, wherein the magnetically attractive layer comprises a ferrous sheet or a steel sheet.

Embodiment 10: The mount of Embodiment 1, wherein the one or more attachment elements comprise three attachment elements disposed in a triangular shape around the body.

Embodiment 11: The mount of Embodiment 10, wherein two of the three attachment elements are used to couple the mount to the tray bail.

Embodiment 12: The mount of Embodiment 10, wherein the three attachment elements comprise: a first attachment element at a first side of the body; a second attachment element horizontally aligned with the first attachment element at a second side of the body; and a third attachment element at a third side of the body.

Embodiment 13: The mount of Embodiment 1, wherein the body engages with a first side of the tray bail and the one or more attachment elements engage with a second side of the tray bail that is opposite of the first side.

Embodiment 14: The mount of Embodiment 1, further comprising: one or more coupling elements for coupling the housing to the remote.

Embodiment 15: The mount of Embodiment 14, wherein the one or more coupling elements engage with one or more receiving elements on the remote to couple the housing to the remote.

What is claimed is:

1. A cradle, the cradle comprising:
a housing having an aperture therethrough for holding a remote that controls one or more aspects associated with operations of a surgical console; and
one or more magnets disposed within the housing, the one or more magnets configured to magnetically interact with a magnetically attractive layer within a tray of the surgical console, and movably couple the housing to the tray.

2. The cradle of claim 1, wherein the housing comprises:
a first casing having a first surface configured to engage with the tray,
wherein a first one or more depressions are formed in the first surface.

3. The cradle of claim 2, wherein the first one or more depressions comprise two depressions horizontally aligned with each other on opposite sides of the first surface.

4. The cradle of claim 2, wherein the first surface comprises:
a first convex portion;
a second convex portion; and
a planar portion between the first convex portion and the second convex portion.

5. The cradle of claim 2, wherein the housing further comprises:
a second casing coupled to the first casing, the second casing having a second surface that faces an opposite direction of the first surface,
wherein a second one or more depressions are formed in the second surface.

6. The cradle of claim 5, wherein the second one or more depressions comprise two depressions horizontally aligned with each other on opposite sides of the second surface.

7. The cradle of claim 5, wherein the second surface comprises a convex shape.

8. The cradle of claim 2, wherein the first casing comprises one or more openings through which one or more elastomeric pads protrude, the one or more elastomeric pads configured to maintain positioning of the housing on the tray at a user-determined location.

9. The cradle of claim 8, wherein at least one of the one or more openings correspond to a positioning of the one or more magnets disposed within the housing.

10. The cradle of claim 8, wherein the one or more openings comprise a plurality of openings disposed equidistantly from each other around a perimeter of the first casing.

11. The cradle of claim 8, wherein:
the one or more elastomeric pads define one or more receptacles, and
at least one of the one or more magnets is disposed in the one or more receptacles.

12. The cradle of claim 1, wherein the housing comprises:
one or more receptacles disposed within the housing, and
wherein the one or more magnets are disposed within the one or more receptacles.

13. The cradle of claim 1, wherein an inner perimeter of the housing engages with one or more surfaces of the remote.

14. The cradle of claim 1, further comprising:
one or more coupling elements for coupling the housing to the remote.

15. The cradle of claim 14, wherein the one or more coupling elements engage with one or more receiving elements on the remote to couple the housing to the remote.
